(19) United States
(12) Patent Application Publication
(10) Pub. No.: US 2024/0124382 A1
(43) Pub. Date: Apr. 18, 2024

(54) IMPROVED PROCESS FOR MANUFACTURING HIGH-PURITY ALKYL ACRYLATES

(71) Applicant: Arkema France, Colombes (FR)

(72) Inventors: Anne MORELIERE, SAINT AVOLD cedex (FR); Serge TRETJAK, SAINT AVOLD cedex (FR); Marc ESCH, SAINT AVOLD cedex (FR); Camille HILPERT, SAINT AVOLD cedex (FR)

(21) Appl. No.: 18/276,675

(22) PCT Filed: Feb. 8, 2022
(86) PCT No.: PCT/FR2022/050228

§ 371 Date: Aug. 10, 2023

(30) Foreign Application Priority Data
Feb. 15, 2021 (FR) 2101402

(51) Int. Cl. C07C 67/08; C07C 67/54

(52) U.S. Cl. CPC C07C 67/08; C07C 67/54

(57) ABSTRACT

The invention relates to the manufacture of alkyl acrylates by direct esterification of acrylic acid with the corresponding alcohol. In particular, the invention relates to the use of a cracker in combination with a topping column equipped with a side draw-off allowing a stream rich in acidic impurities such as β-hydroxypropionic acid and β-acryloxypropionic acid to be drawn off during distillation of the crude reaction mixture, so as to produce an acrylic ester meeting purity standards compatible with its use for preparing acrylic polymers.

### TECHNICAL FIELD

The present invention relates to the manufacture of alkyl (meth)acrylates by direct esterification of (meth)acrylic acid with the corresponding alcohol.

The subject is an improved process for the manufacture of C₄-C₁₀ alkyl (meth)acrylate, in particular 2-ethylhexyl acrylate, comprising a step of upgrading the heavy byproducts generated during this manufacture, leading to high productivity of a product meeting the purity and acidity standards, under optimized energy conditions.

### TECHNICAL BACKGROUND AND PROBLEM

The esterification of (meth)acrylic acid is an equilibrated reaction with generation of water, which it is necessary to remove during the reaction in order to shift the equilibrium in the direction of the production of the (meth)acrylic ester.

The problems encountered in the manufacture of C₄-C₁₀ alkyl (meth)acrylates by direct esterification of (meth)acrylic acid, generally in the presence of a cationic resin as catalyst, are most often linked to the complexity of the purification steps required after the reaction step to obtain a high-purity product, generally to the detriment of the process productivity.

“Heavy” compounds resulting from Michael addition reactions form spontaneously in the (meth)acrylic monomer production units. These parasitic reactions are favored at the high temperatures encountered in particular in the distillation column bottoms of these units.

This point is illustrated, for example, by the synthesis of 2-ethylhexyl acrylate. The industrial process consists in esterifying acrylic acid with excess 2-ethylhexanol in the presence of an ion exchange resin. At the end of the reaction, the reaction mixture comprises 2-ethylhexyl acrylate, residual acrylic acid, excess 2-ethylhexanol, the stabilizers conventionally used for inhibiting polymerization reactions, and various impurities resulting from side reactions.

Thus, acrylic acid, unreacted 2-ethylhexanol and the reaction water add to the double bond of 2-ethylhexyl acrylate to form, mainly:
2-ethylhexyl acryloxypropionate (2EHAP), by the addition of acrylic acid to 2-ethylhexyl acrylate;
2-ethylhexyl hydroxypropionate (2EHHP) by the addition of water to 2-ethylhexyl acrylate;
2-ethylhexyl 2-ethylhexyloxypropionate (OPO) by addition of 2-ethylhexanol to 2-ethylhexyl acrylate.

Polyaddition or the formation of mixed compounds is also possible.

Other heavy byproducts formed during the synthesis of 2-ethylhexyl acrylate are olefins, such as 2-ethylhex-2-ene and 3-methylhept-2-ene or 3-hydroxypropanoic acid (HPA) by hydrolysis of the acrylic acid dimer (di-AA) with the reaction water.

One of the features of Michael adducts is that their boiling point is above the boiling points of acrylic acid (AA), 2-ethylhexanol and 2-ethylhexyl acrylate (2EHA). As their volatility is low, they accumulate at the bottom of the last distillation column, at the bottom of the evaporator used for concentrating this residue.

In addition to these heavy byproducts and a few percent of free monomers, the evaporator residue also contains a high concentration of polymerization inhibitors accumulated in the course of the purification steps, such as phenothiazine in its free form or as an adduct of AA or 2EHA, and also compounds of polymeric nature that are more or less soluble in the medium. In general, this residue is often eliminated by incineration, resulting in a significant loss of yield. These byproducts present a problem of losses of starting materials and a problem of separation in order to be easily removed, all the more so as they can form azeotropes with the desired ester.

It is therefore difficult to achieve a (meth)acrylic ester of high purity.

In addition, most fields of application, notably that of pressure-sensitive adhesives (PSA), require the preparation of (meth)acrylic polymers from monomers which meet strict purity standards (>99.7%) and which are free of acidity, in particular including a content of acid-based impurities (HPA+di-AA or AA) of less than 90 ppm.

To limit these impurities, EP 3 174 844 describes the use of an excess of alcohol and the circulation of a reaction loop comprising the esterification reactor and a distillation column removing the water produced in the form of an azeotrope with the esterifying alcohol. This process results in a purified ester containing low traces of acid-based impurities.

To solve this problem, WO 2020/234519 describes the use of a distillation column equipped with a side draw-off and placed at the outlet of the reaction section; this allows a solution concentrated in HPA and acrylic acid dimers to be purged continuously from the side, and thus reduces the amount of residual acidic impurities in the purified product. However, said document makes no mention of the fate of the olefins in this implementation.

There is thus still a need to improve the removal of acidic impurities, in particular the removal of 3-hydroxypropanoic acid (HPA), and also olefins, in the acrylic ester synthesis/purification processes described in the prior art.

It has now been discovered that the use of thermal cracking, with or without a catalyst, allows Michael adducts to be converted in high yield, without solid deposits in the facility, in a process allowing the production of a high-purity C₄-C₁₀ ester, in particular 2-ethylhexyl acrylate.

One subject of the present invention is thus a process for the recovery/purification of alkyl acrylate, which is simple to perform, leading to a product which meets purity standards with optimized productivity, while at the same time limiting the size of the equipment to be used and the energy cost.

### SUMMARY OF THE INVENTION

One subject of the invention is a process for recovering/purifying a C₄-C₁₀ acrylic ester from a crude reaction mixture obtained via the direct esterification of acrylic acid with the corresponding alcohol, in which a stream rich in acidic impurities such as β-hydroxypropionic acid and β-acryloxypropionic acid is withdrawn via a side outlet during distillation of the crude reaction mixture, characterized in that it comprises a step of thermal cracking of the residues at the bottom of the purification column, leading to the production of cracking products which are recycled into the process.

The process according to the invention describes in detail the particular case of synthesizing 2-ethylhexyl acrylate.

Advantageously, the cracking products are reintroduced outside the reaction zone, thus eliminating the negative effects of the olefins on acrylic acid losses and avoiding the formation on the catalytic resin of 2-ethylhexyl hydroxypropionate (2EHHP) by esterification of 3-hydroxypropanoic acid (HPA) with 2-ethylhexanol.

The term “stream rich in acidic impurities” means that the bulk of these acidic impurities generated during the esterification reaction are present in the stream which is drawn off laterally from the distillation column fed with the crude reaction mixture. This stream comprises, in addition to the acrylic ester, traces of unreacted reagents and heavy byproducts with a boiling point greater than that of the acrylic ester, and also traces of water.

The stream rich in acidic impurities drawn off may be in gaseous form or in liquid form, preferably in liquid form.

The side draw-off is preferably performed at a lower level than the level of feeding of the distillation column, which makes it possible to minimize the presence of the upgradable reagents, such as acrylic acid and the esterifying alcohol, in the drawn-off stream.

According to one embodiment of the process according to the invention, the stream rich in acidic impurities, drawn off laterally, is subjected to a treatment with water, in order to separate out said acidic impurities and to recycle the treated stream into the distillation column.

According to one embodiment, the treated stream free of the bulk of the acidic impurities is recycled into the distillation column.

The treated stream free of the bulk of the acidic impurities can be recycled into the distillation column at a lower level or at a higher level than the side draw-off; preferably, it is recycled at a higher level than the side draw-off.

Preferably, the treated stream free of the bulk of the acidic impurities is recycled into the distillation column at a lower level than the distillation column feed level.

The process according to the invention is performed using a purification system comprising at least one distillation column equipped with a side draw-off allowing the separation of the bulk of the acidic impurities present in the crude reaction mixture. Preferably, said distillation column is a topping column separating the light compounds, such as the unreacted reagents present in the reaction medium, at the top.

Another subject of the invention is a process for the recovery/purification of a C₄-C₁₀ acrylic ester from a crude reaction mixture obtained by direct esterification of acrylic acid with the corresponding alcohol, comprising at least the following steps:
i) the reaction mixture is subjected to topping in a distillation column equipped with a side draw-off making it possible to obtain:
   at the top, a stream composed essentially of the unreacted reagents;
   at the bottom, a stream comprising the desired ester and heavy byproducts;
   by side draw-off, a stream rich in acidic impurities;
ii) the bottom stream from the topping column is subjected to a rectification column making it possible to separate:
   at the top, the purified desired ester;
   at the bottom, a stream containing heavy byproducts, which is concentrated on a film evaporator or distilled in a tailing column so as to recycle the light compounds present to the topping column, and to eliminate a final residue of heavy byproducts;
iii) the bottom stream of the rectification column is subjected to a thermal or thermal and catalytic treatment performed in a reactor (cracker) placed at the outlet of the evaporator, making it possible to separate:
   at the top, a stream of upgradable products recycled separately or mixed with the evaporator head stream;
   at the bottom, a residue sent to a treatment plant.

The process according to the invention may also comprise a step iv) of treating the side draw-off stream:
iv) the stream rich in acidic impurities is subjected to a step of washing with an aqueous stream, which produces, after decantation:
   an aqueous phase comprising all of the acidic impurities, which can be sent to a biological treatment plant or partly used as aqueous washing stream, and
   an organic phase comprising the desired ester, heavy byproducts and traces of water and of reagents, which is at least partly recycled into the topping column.

The process according to the invention may also comprise a step v) of treating the head stream of the thermal or thermal-catalytic treatment reactor (cracker):
v) the cracker head stream is subjected to a step of washing with an aqueous stream, which produces, after decantation:
   an aqueous phase comprising all of the acidic impurities, which can be sent to a biological treatment plant or partly used as aqueous washing stream, and
   an organic phase, comprising the desired ester, alcohol and acid, and also heavy byproducts and traces of water, which is at least partly recycled into the topping column feed or into the side draw-off washing step.

The process according to the invention allows a C₄-C₁₀ acrylic ester to be obtained with a purity of greater than or equal to 99.7%, or even greater than 99.8%, and with a content of acidic impurities (HPA+AA+AA dimer) of less than 90 ppm, and finally a water content of less than 400 ppm, incorporating a thermal or thermal and catalytic process to perform the cracking of Michael adducts into reagents (acrylic acid and alcohol) and finished product, thus increasing the productivity of the process by limiting the amount of residue to be eliminated.

The invention advantageously applies to the production of 2-ethylhexyl acrylate or 2-octyl acrylate, meeting the purity standards required for the production of polymers that may be used, for example, in the field of adhesives or coatings.

Another subject of the invention is a process for producing a C₄-C₁₀ acrylic ester free of acidic impurities and olefins, by direct esterification of acrylic acid with the corresponding alcohol, comprising the recovery/purification process as defined above.

Advantageously, said thermal or thermal and catalytic treatment allows the Michael adducts to be upgraded in a process allowing the production of a C₄-C₁₀ ester, in particular 2-ethylhexyl acrylate, by combining a reactor allowing cracking of the purge products from the bottom of the evaporator placed at the bottom of the 2-ethylhexyl acrylate purification column, with optionally an additional decanter according to the various modes of invention allowing recycling of the head products resulting from cracking.

Thus, the cracker head products may be processed according to various embodiments of the invention:
washed in a decanter with water, then mixed with the evaporator head product,
washed in a decanter with water, then introduced into the decanter already present at the side draw-off,
introduced directly into the decanter already present on the side draw-off.

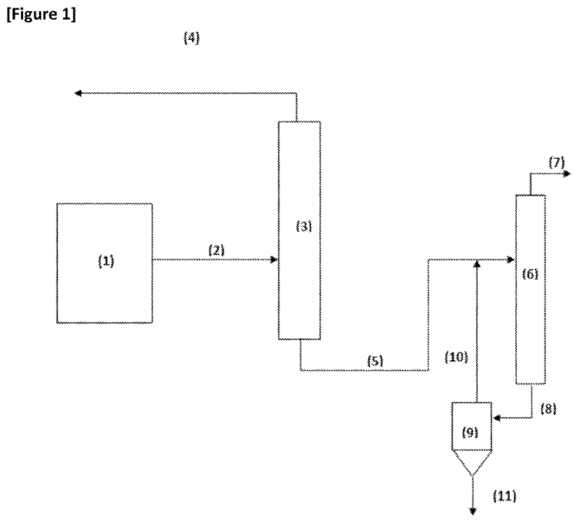

Other features and advantages of the invention will emerge more clearly on reading the following detailed description, with reference to the appended FIGS. 1 to 7, which represent:

FIG. 1: schematic diagram of a first two-column purification process described in EP 3 174 844.

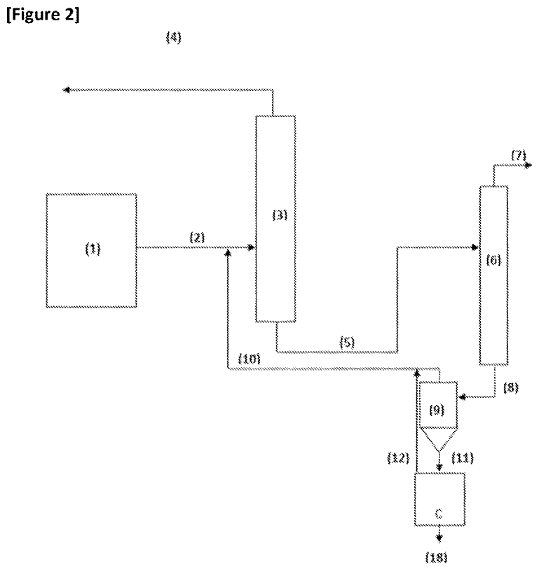

FIG. 2: schematic diagram of a second two-column purification process described in EP 3 174 844, to which a cracker C is added.

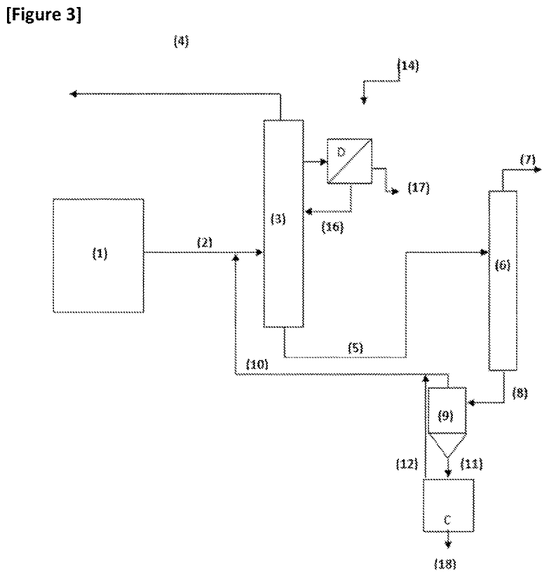

FIG. 3: schematic diagram of the process according to the invention, with the addition of a cracker C and recycling of the cracker head stream to the topping column inlet.

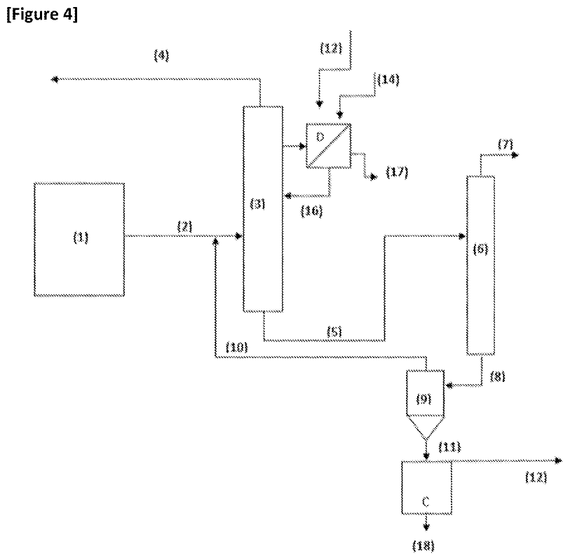

FIG. 4: schematic diagram of an embodiment of the process according to the invention, with recycling of the cracker head stream into the decanter D located on the side draw-off of the topping column.

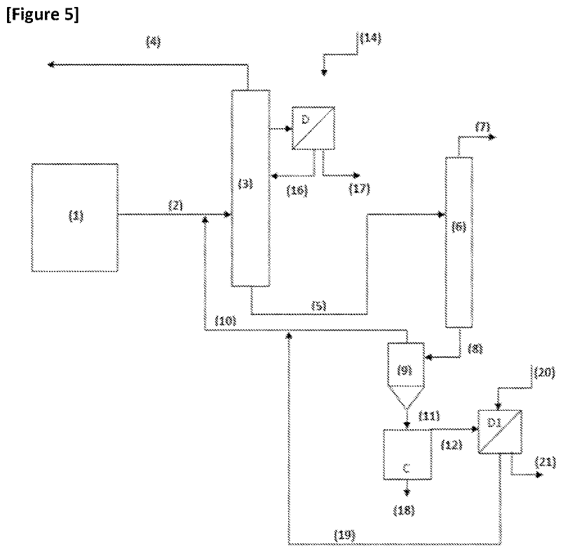

FIG. 5: schematic diagram of an embodiment of the process according to the invention, with recycling of the cracker head stream C into a decanter D1, followed by recycling of the washed product to the inlet of the topping column.

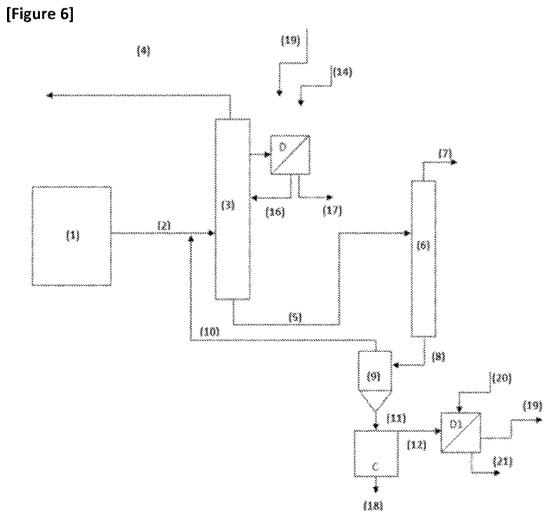

FIG. 6: schematic diagram of an embodiment of the process according to the invention, with recycling of the cracker head stream C into a decanter D1, followed by recycling of the washed product into the decanter D located on the side draw-off of the topping column.

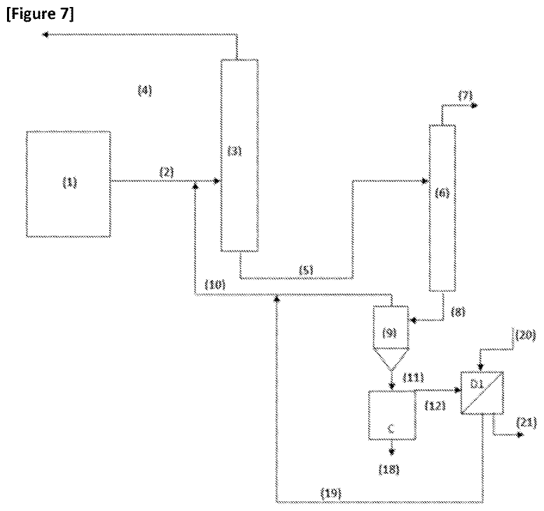

FIG. 7: schematic diagram of an embodiment of the process according to the invention, with recycling of the cracker head stream into a decanter D1, followed by recycling of the washed product to the inlet of the topping column.

### DETAILED DESCRIPTION OF THE INVENTION

According to a first aspect, the invention relates to a process for recovering/purifying a C₄-C₁₀ acrylic ester from a crude reaction mixture obtained by direct esterification of acrylic acid with the corresponding alcohol, in which a stream rich in acidic impurities such as β-hydroxypropionic acid and β-acryloxypropionic acid is withdrawn via a side outlet during the distillation of the crude reaction mixture, characterized in that it comprises a step of thermal cracking of the residues at the bottom of the purification column, leading to the production of cracking products which are recycled into the process.

Advantageously, said cracking products which are recycled into the process are the ester, the reagents (alcohol, acids), but also olefins such as 2-ethylhex-2-ene and 3-methylhetp-2-ene and acidic impurities which must be removed.

In the process according to the invention, the decomposition of the Michael adducts may be performed in continuous, semi-continuous or batch mode. Continuous operation is the preferred mode of operation for this esterification process. A tubular reactor, a jacketed stirred reactor or a reactor with an external heating loop with forced circulation or natural circulation (thermosiphon type) may be used. The upgradable compounds generated by the cracking reaction are collected after condensation of the vapors at the top of the reactor or at the top of a distillation column mounted on the reactor.

The reaction temperature and pressure in the reactor are connected in such a way that the reagents, such as acrylic acid or 2-ethylhexanol, or the final product are removed by evaporation, while at the same time maintaining the Michael adducts, such as 2-ethylhexyl hydroxypropionate (2EHHP), in the reaction medium.

The decomposition reaction is performed in a temperature range from 180 to 280° C., and more especially from 200° C. to 250° C. The pressure maintained above the reactor is between 10 000 pascals and atmospheric pressure, more especially between 30 000 and 60 000 pascals. In the process according to the invention, the decomposition of the Michael adducts may be performed in the presence or absence of a protic acid catalyst such as sulfuric acid or para-toluenesulfonic acid (PTSA). The absence of catalyst is preferred, as it avoids creating a special catalyst introduction line in this cracking reactor and, above all, avoids complicating the thermal upgrading treatment of the final residue due to the presence of this strong acid concentrated to a scale of a few percent.

The residence time based on the feed rate (kg/h) relative to the reaction volume (1) ranges between 0.5 and 20 hours, preferably between 1 and 7 hours.

The head product from this cracker consists of 2-ethylhexyl acrylate, the starting reagents, but also olefins and HPA, which will necessitate washing with water followed by continuous decantation in the static or centrifugal decanter placed on the side draw-off of the column, or by using additional equipment placed directly at the outlet of this cracker.

The invention is based on purging a stream rich in acidic impurities using a side draw-off preferably fitted to a topping column in a process for purifying a crude reaction mixture obtained via the direct esterification of acrylic acid with a C₄-C₁₀ alcohol, and also a reactor for upgrading the Michael adducts formed in this esterification process.

The esterifying alcohol may be a primary or secondary aliphatic alcohol, including a linear or branched alkyl chain containing from 4 to 10 carbon atoms. Examples of alcohols that may be mentioned include butanol, 2-ethylhexanol, n-octanol, 2-octanol, n-decanol and 2-propylheptanol.

Preferably, the alcohol is 2-ethylhexanol or 2-octanol.

The esterification reaction is generally performed in a reactor on which is mounted a distillation column for extracting the water generated by the reaction. The reaction water is removed as it is formed in the form of an azeotrope with the esterifying alcohol so as to shift the esterification equilibrium.

The operating conditions of the esterification reaction are not critical, it being possible for the process according to the invention to be applied to the reaction mixture irrespective of the process for obtaining it. Thus, the reaction can be performed in an excess of acid or an excess of alcohol, at a temperature generally between 70° C. and 100° C., preferably between 75° C. and 95° C.

The reactor may be a fixed bed reactor or a slurry bed reactor. The distillation column mounted on the reactor is generally a packed column and it is equipped with a top condenser and a decanter, making it possible to separate by settling the vapors condensed at the top and to separate an organic phase comprising alcohol and traces of ester, which is recycled into the column, and an aqueous phase, which is removed. The column generally operates at a pressure ranging from 6000 to 12 000 pascals.

With reference to FIG. 1, which represents the schematic diagram of a prior art acrylic ester recovery/purification process described in EP 3 174 844, the reaction mixture leaving the reactor feeds the topping column (3) which essentially separates the unconverted reagents (4) from the ester and heavy impurities (5) which feed the purification column (6), which allows the purified ester to be obtained at the top (7) and a stream (11) left over from this process after treatment of the bottom of the column (6) on an evaporator (9).

With reference to FIG. 2, which represents the schematic diagram of a second, two-column acrylic ester recovery/purification process described in EP 3 174 844, to which a cracker D1 has been added. The reaction mixture leaving the reactor feeds the topping column (3) which essentially separates the unconverted reagents (4) from the ester and heavy impurities (5) which feed the purification column (6), which allows the purified ester to be obtained at the top (7) and a stream (11) left over from this process after treatment of the bottom of the column (6) on an evaporator (9). Finally, the stream (11) feeds a cracking reactor C. The head stream of the latter is recycled (12) to the inlet of column (3).

The invention overcomes the drawbacks of said prior art processes, by using, in addition to a distillation column equipped with a side draw-off as a topping column (3), a cracker, leading to the production of cracking products which are reintroduced into the process.

FIG. 3 illustrates an embodiment of the process according to the invention. The reaction mixture leaving the reactor feeds the topping column (3), which essentially separates the unconverted reagents (4) from the ester and heavy impurities (5), and also a side draw-off which allows the acidic impurities to be treated by washing this stream with water (14), separating by settling the aqueous and organic phases in decanter D and then recycling the organic phase into column (3). The bottom of column (3) feeds the purification column (6), which allows the purified ester to be obtained at the top (7), and a stream (11) left over from this process after treatment of the bottom of column (6) on an evaporator (9). Finally, stream (11) is fed to a cracking reactor C. The head stream from said reactor is recycled (12) together with the head stream from the evaporator (10) into the inlet of column (3).

The cracker bottom residue 18 is sent to a treatment plant.

According to the schematic diagram of the process according to the invention, represented in FIG. 4, a thermal or thermal and catalytic cracker (13) is placed at the outlet of evaporator 9, allowing the bottom of finishing column 6 to be concentrated. This cracker is fed with stream 11. The cracker bottom residue 18 is sent to a treatment plant. The upgradable product stream is recycled into decanter D, which is located on the draw-off line 15 of column 3. Streams 12 and 15 are then washed with water 14. The aqueous phase is then sent to a biological plant, while the organic stream 16 is recycled into column 3.

With reference to FIG. 4, which represents the preferred embodiment of the invention, the topping column (3) includes an equivalent of 10 and 30 theoretical trays, preferably 15 to 20 theoretical stages. The inserts used for the column may be valve trays or perforated weir trays, crossflow trays such as Dual Flow Trays, Ripple Trays, Turbo Grid Shells, or ordered packing, for instance structured packing such as Mellapack 250X from Sulzer.

The feed to the topping column consists of the stream from the reaction loop catalyzing the esterification reaction, preferably with a strong cationic resin, for example a sulfonated resin of styrene/divinyl benzene type bearing sulfonic groups. For example, mention may be made of the products sold under the name Lewatit K2620 or K2621 by the company Lanxess, or those sold under the name Amberlyst A15, A16 or A46 by the company Rohm & Haas.

The feed (2) to the topping column takes place in the upper third of this column, preferably between theoretical trays 3 to 10 counting from the top of the column.

Stream (4) at the top of column (3) essentially comprises the unreacted reagents. This upgradable stream (4) is recycled into the reaction.

The column operates with a reflux ratio (flow of condensed liquid returned to the column/flow (4)) of between 1/5 and 1/1, preferably 1/3.

The side draw-off stream 15 may be gaseous or liquid, preferably liquid. The draw-off is located between theoretical trays 5 to 15, preferably between 8 and 12 counting from the top of the column. The location of this side draw-off is judiciously chosen so as to maximize the concentration of HPA and di-AA while at the same time minimizing the presence of upgradable reagents (AA and 2-ethylhexanol). Needless to say, this side draw-off includes the amount of stabilizers required for fouling-free operation. If need be, in the event of gas-phase draw-off, another stabilizer can also be added. Advantageously, from 100 to 5000 ppm of polymerization inhibitor are introduced into the purification system according to the process of the invention.

Examples of useful polymerization inhibitors that may be mentioned include phenothiazine (PTZ), hydroquinone (HQ), hydroquinone monomethyl ether (HQME), di-tert-butyl-para-cresol (BHT), para-phenylenediamine, TEMPO (2,2,6,6-tetramethyl-1-piperidinyloxy), di-tert-butylcatechol, or TEMPO derivatives, such as OH-TEMPO, alone or as mixtures in any proportion, at contents in the reaction medium which may be between 50 ppm and 5000 ppm, optionally in the presence of depleted air, but generally at contents of between 150 ppm and 1000 ppm. Polymerization inhibitors may be added at various points, with the introduction of the reagents or at the top of the distillation column.

After cooling to the temperature at which decantation is to take place, preferably between 20° C. and 70° C., water (14) is added in a proportion of between 5% and 100%, preferably between 30% and 80%, relative to the stream coming from the side draw-off (15). The head product from the cracker (12), cooled to between 20° C. and 70° C., is also introduced into this decanter D. The washed organic stream (16) is returned to the column between theoretical trays 5 to 15, preferably 7 to 12. This organic stream contains the olefins formed, which are removed at the top of column 3. When mixed, the olefin concentration at the top of this column varies only slightly (3500 ppm to 4000 ppm), and therefore does not interfere with the functioning of the process. The aqueous phase containing the acid-based impurities may be either partially or totally recycled into stream (14) or discharged to a biological plant.

To make the inhibitors more efficient, oxygen, air or depleted air containing 7% 02 can be injected into the bottom of the column. Preferably, the amount of oxygen injected corresponds to a content of 0.2% to 0.5% relative to the amount of organic vapor in the column.

The column may be operated under vacuum, so as to minimize the thermal exposure of the heat-sensitive compounds within the column. Advantageously, column (3) operates under a vacuum ranging from 1333 to 13 332 pascals.

Stream (5) drawn off at the bottom of this column and fed to the column for production of the desired ester (6) includes at least 92% by weight of the desired ester, acid-based impurities (HPA, di-AA), alcohol-based impurities and Michael adducts. This stream preferably feeds the column (6) between theoretical tray 1 to 3 counting from the bottom of the column (6).

The purge column (6) includes an equivalent of 2 and 15 theoretical trays, preferably 5 to 10 theoretical stages.

Column (6) is, for example, a perforated-tray or packed column. The inserts used for the column may be valve trays or perforated weir trays, or crossflow trays such as Dual Flow Trays, Ripple Trays, Turbo Grid Shells, or ordered packing, for instance structured packing such as Mellapack 250X from Sulzer.

The head stream (7) from column (6) consists of the desired ester, having the following specifications: ester purity >99.7%, content of acidic impurities (HPA+diAA+AA)<90 ppm and finally a water content <400 ppm.

The column operates with a reflux ratio (flow of condensed liquid returned to the column/flow (7)) of between 1/5 and 1/1, preferably 1/5 to 1/2. Like column (3), it is stabilized and air or depleted air (7% 02) is injected at the bottom of the column. The column may be operated under vacuum, so as to minimize the thermal exposure of the heat-sensitive compounds within the column. Advantageously, column (6) operates under a vacuum ranging from 1333 to 13 332 Pa.

Advantageously, the operating temperature is between 50° C. and 160° C.

The bottom stream (8) is concentrated on a scraped-film evaporator (9) so as to recycle the light compounds present into the start of the purification section upstream of column (3) or column (6), and allows the residue (11) of heavy products to be eliminated. This residue is fed into a forced recirculation reactor (13) comprising an external exchanger heated with steam at 33×10⁵ pascals. The temperature of the reaction medium is between 1800 and 250° C., preferably 230° C. to 250° C. The pressure in this reactor is maintained between 13 000 Pa and atmospheric pressure, preferably between 39 000 Pa and 78 000 Pa, by means of a liquid ring pump or venturi pump. The bottom product constitutes the final residue and is sent to the appropriate channel. The top product (12), condensed at a temperature of 20° C. to 30° C., is sent to decanter D, located on the side draw-off of column (3). There is no need to inject air or depleted air into this reactor, as product (11) contains all the stabilizers used in the process.

With reference to FIG. 5, a further embodiment is described in which a thermal or thermal and catalytic cracker (13) is placed at the outlet of evaporator 9, allowing the bottom of the finishing column 6 to be concentrated. This cracker is fed with stream 11. The cracker bottom residue 18 is sent to a treatment plant. The upgradable product stream is recycled into decanter D1, washed with water 20, then the organic phase is mixed with stream 10 and recycled into the inlet of column 3. The aqueous stream (21) may be sent to a water treatment plant or recycled into the reaction to upgrade the AA by esterification.

With reference to FIG. 6, another embodiment is described in which a thermal or thermal and catalytic cracker (13) is placed at the outlet of the evaporator (9), allowing the bottom of the finishing column (6) to be concentrated. This cracker is fed with stream 11. The cracker bottom residue 18 is sent to a treatment plant. The upgradable product stream is recycled into decanter D1, washed with water (20) and then the organic phase is recycled into decanter D, in which it is washed with water (14) at the same time as the draw-off stream from column 3. The aqueous stream (21) may be sent to a water treatment plant or recycled into the reaction to upgrade the AA by esterification.

With reference to FIG. 7, another embodiment is described in which a thermal or thermal and catalytic cracker (13) is placed at the outlet of evaporator 9, allowing the bottom of the finishing column 6 to be concentrated. This cracker is fed with stream 11. The residue (18) from the bottom of the cracker is sent to a treatment plant. The upgradable product stream is recycled into decanter D1, washed with water (20) and the organic phase is then recycled into the inlet of the topping column.

The examples below illustrate the present invention without, however, limiting the scope thereof.

### EXPERIMENTAL SECTION

In the examples, the percentages are shown by weight, unless otherwise indicated, and the following abbreviations were used:
AA: acrylic acid
2EHA: 2-ethylhexyl acrylate
E2OH: 2-ethylhexanol
Di-AA: AA dimers
2EHHP: 2-ethylhexyl hydroxypropionate
2EHAP: 2-ethylhexyl acryloxypropionate
OPO: 2-ethylhexyl 2-ethylhexyloxypropionate
HPA: hydroxypropionic acid
2EHEXENE: 2-ethylhexene
WATER
AIR
In order to compare the prior art with the invention, columns 3,6 have a fixed configuration.
The main characteristics of these two columns are as follows:
Column 3: 45 Dual Flow trays
   Column head pressure: 3333 pascals
   Feed: tray 15
   Air: tray 45
   Stabilizer: tray 1
Column 6: 26 Dual Flow trays
   Column head pressure: 2666 pascals
   Feed: tray 25
   Air: tray 26
   Stabilizer: tray 1

### Example 1: 2EHA Process (FIG. 1)

Feed (2) contains 70 ppm of HPA.

The facility in this basic case allows the production of 4950 kg/h, i.e. about 119 t/d to commercial specifications, with a 2EHA purity of 99.7% and a content of acidic compounds (di-AA+AA+3HPA) of 84 ppm.

The results obtained are presented in Table 1.

TABLE 1

| | | Stream | | | | |
|---|---|---|---|---|---|---|
| | | 2 | 4 | 5 | 7 | 11 |
| Temperature | C. | 115 | 30 | 143 | 24 | 130 |
| Pressure | Pascals | 43 000 | 4 × 10⁵ | 1.5 × 10⁴ | 3 × 10⁵ | 4000 |
| Mass flow rate | kg/hr | 13 650 | 7620 | 6300 | 4950 | 440 |
| Test 2 | | | | | | |
| Mass fraction | | | | | | |
| AA | | 0.065 | 0.119 | 0.000 | 0.000 | 0.000 |
| E2HOH | | 0.233 | 0.425 | 0.000 | 0.000 | 0.000 |
| 2EHA | | 0.651 | 0.402 | 0.952 | 0.997 | 0.487 |
| WATER | | 0.005 | 0.008 | 0.000 | 0.000 | 0.000 |
| DIAA | ppm | 23.0 | 4.2 | 500.0 | 29.0 | 2565 |
| 2EHEXENE | ppm | 1860.0 | 3380.0 | 0.0 | 0.0 | 0.0 |
| HPA | ppm | 71.0 | 91.0 | 0.0 | 55 | 15 |

### Example 2: 2EHA Process (FIG. 2)

In this example, a cracker is used to treat stream 11. This time, stream 12 representing the cracker head (13) is reinjected with stream 10 from evaporator head 9 into the feed of column 3.

The configuration as envisaged does not allow an amount of acidic compounds (di-AA+AA+3HPA)<90 ppm to be obtained. The product is thus out of specification, with a very high HPA content.

The results obtained are presented in Table 2.

TABLE 2

| | | Stream | | | | | | |
|---|---|---|---|---|---|---|---|---|
| | | 2 | 4 | 5 | 7 | 10 | 11 | 12 |
| Temperature | ° C. | 115 | 30 | 143 | 24 | 35 | 130 | 20 |
| Pressure | Pascals | 43 000 | 4 × 10⁵ | 2 × 10⁴ | 3 × 10⁵ | 2 × 10⁵ | 4000 | 3 × 10⁵ |
| Mass flow | kg/hr | 13 850.0 | 7620.0 | 6500.0 | 5150.0 | 913.7 | 436.3 | 300.0 |
| rate | | | | | | | | |
| mass % | | | | | | | | |
| AA | | 0.066 | 0.123 | 0.000 | 0.000 | 0.000 | 0.000 | 0.118 |
| E2HOH | | 0.230 | 0.426 | 0.000 | 0.000 | 0.000 | 0.000 | 0.110 |
| 2EHA | | 0.653 | 0.397 | 0.953 | 0.996 | 0.934 | 0.487 | 0.729 |
| WATER | | 0.005 | 0.009 | 0.000 | 0.000 | 0.000 | 0.000 | 0.010 |
| DIAA | ppm | 223.00 | 4.00 | 480.00 | 31.00 | 2032.00 | 2519.00 | 0.00 |
| 2EHEXENE | ppm | 2460.00 | 4529.00 | 0.00 | 0.00 | 0.00 | 0.00 | 30000 |
| HPA | ppm | 135.00 | 145.00 | 124.00 | 145.00 | 41.00 | 40.00 | 2991.00 |

### Example 3: 2EHA Process According to the Invention (FIG. 4)

The cracker head stream 12 is recycled into the decanter D located on the side draw-off. The amount of water (14) used to wash stream 12 and product 15 extracted from the column was set at 500 kg/h. The acidity of the purified ester (HPA+diAA) remains well below <90 ppm. Moreover, the ester produced by the cracking process is upgraded as a final product, since the production rate of purified ester increases from 4950 kg/h to 5150 kg/h, i.e. an increase in production of 5%.

The Michael adducts can thus be upgraded while at the same time maintaining the very high purity of the end product by increasing the production rate.

The results obtained are presented in Table 3.

TABLE 3

| | | Stream | | | | | |
|---|---|---|---|---|---|---|---|
| | | 12 | 5 | 2 | 14 | 17 | 11 |
| Temperature | C. | 20 | 143 | 115 | 20 | 20 | 130 |
| Pressure | Pascals | 10⁵ | 2 × 10⁴ | 43 000 | 10⁵ | 10⁵ | 4000 |
| Mass flow rate | kg/hr | 300 | 6500 | 13 850 | 500 | 514 | 438 |
| Mass fraction | | | | | | | |
| AA | | 0.118 | 0.000 | 0.065 | 0.000 | 0.027 | 0.000 |
| E2HOH | | 0.110 | 0.000 | 0.233 | 0.000 | 0.000 | 0.000 |
| 2EHA | | 0.729 | 0.954 | 0.651 | 0.000 | 0.000 | 0.487 |
| WATER | | 0.010 | 0.000 | 0.005 | 1.000 | 0.961 | 0.000 |
| DIAA | ppm | 0.0 | 464.0 | 220.0 | 0.0 | 126.0 | 2436.0 |
| 2EHEXENE | ppm | 29 910 | 0.0 | 1871.0 | 0.0 | 15.0 | 0.0 |
| HPA | ppm | 2991.0 | 17.0 | 70.0 | 0.0 | 2810.0 | 5.5 |

| | Stream | | | | |
|---|---|---|---|---|---|
| | 7 | 4 | 15 | 10 | 16 |
| Temperature | 24 | 30 | 139 | 35 | 20 |
| Pressure | 3 × 10⁵ | 4 × 10⁵ | 10 000 | 2 × 10⁵ | 10⁵ |
| Mass flow rate | 5150 | 7905 | 500 | 912 | 786 |
| Mass fraction | | | | | |
| AA | 0.000 | 0.120 | 0.000 | 0.000 | 0.027 |
| E2HOH | 0.000 | 0.420 | 0.002 | 0.000 | 0.043 |
| 2EHA | 0.997 | 0.406 | 0.956 | 0.934 | 0.887 |
| WATER | 0.000 | 0.009 | 0.000 | 0.000 | 0.011 |
| DIAA | 30.0 | 4.0 | 167.0 | 1965 | 24.0 |
| 2EHEXENE | 0.0 | 4440 | 1.3 | 0.0 | 11 416 |
| HPA | 20.0 | 43.0 | 1167.0 | 5.8 | 46.0 |

### Example 4: 2EHA Process According to the Invention (FIG. 6)

In this example, a first wash and decantation (D1) is performed on the stream from the cracker head (12), then this product is fed to the decanter (D) located on the side draw-off of column 3.

The acidity of the purified ester (HPA+diAA) remains well below <90 ppm. Moreover, the ester produced by the cracking process is upgraded as a final product, since the production rate of purified ester increases from 4950 kg/h to 5150 kg/h, i.e. an increase in production of 5%.

This solution, using two decanters, gives the same performance as that described in Example 3.
The results obtained are presented in Table 4.

TABLE 4

| | | Stream | | | | | | | |
|---|---|---|---|---|---|---|---|---|---|
| | | 5 | 2 | 8 | 12 | 14 | 20 | 17 | 11 |
| T | C. | 142.9 | 115.0 | 142.8 | 20.0 | 20.0 | 20.0 | 20.0 | 130.0 |
| Mass flow rate | kg/hr | 6500 | 13 850 | 1350 | 300.0 | 300 | 200 | 304 | 437 |
| Mass fraction | | | | | | | | | |
| AA | | 0.000 | 0.065 | 0.000 | 0.118 | 0.00 | 0.00 | 0.00 | 0.00 |
| E2HOH | | 0.000 | 0.233 | 0.000 | 0.110 | 0.00 | 0.00 | 0.00 | 0.00 |
| 2EHA | | 0.954 | 0.651 | 0.789 | 0.73 | 0.00 | 0.00 | 0.00 | 0.49 |
| WATER | | 0.000 | 0.005 | 0.000 | 0.01 | 1.00 | 1.00 | 0.98 | 0.000 |
| DIAA | ppm | 462 | 220 | 2113 | 2999 | 0 | 0 | 223 | 2431 |
| 2EHEXENE | ppm | 0 | 1870 | 0 | 1 | 0 | 0 | 0 | 0 |
| HPA | ppm | 18 | 70 | 6 | 2991 | 0 | 0 | 2047 | 6 |

| | Stream | | | | | | |
|---|---|---|---|---|---|---|---|
| | 7 | 4 | 21 | 15 | 10 | 19 | 16 |
| T | 23.8 | 29.7 | 20.0 | 139.0 | 35.0 | 20.0 | 20.0 |
| Mass flow rate | 5150 | 7925 | 189 | 500 | 912 | 311.1 | 495.9 |
| Mass fraction | | | | | | | |
| AA | 0.000 | 0.120 | 0.04 | 0.00 | 0.00 | 0.085 | 0.000 |
| E2HOH | 0.000 | 0.419 | 0.00 | 0.02 | 0.00 | 0.107 | 0.002 |
| 2EHA | 0.997 | 0.405 | 0.00 | 0.96 | 0.93 | 0.70 | 0.964 |
| WATER | 0.000 | 0.011 | 0.95 | 0.000 | 0.00 | 0.077 | 0.002 |
| DIAA | 30 | 4 | 0 | 165 | 1 | 2880 | 28 |
| 2EHEXENE | 0 | 4420 | 77 | 0 | 0 | 28 806 | 0 |
| HPA | 21 | 46 | 419 | 1254 | 6 | 334 | 9 |

### Example 5: 2EHA Process According to FIG. 7

In this case, the effect of washing and decantation of the cracker head stream prior to injection into the column 3 feed stream was evaluated. This decanter allows the additional traces of HPA introduced via the cracking process to be removed. Indeed, the amount of HPA present in the purified ester is the same as that of the basic process not using a cracker (Example 2). However, there is the benefit of the increase in productivity from 4950 kg/h to 5150 kg/h due to the additional product supplied by the cracker.

The results obtained are presented in Table 5.

TABLE 5

| | | Stream | | | | | |
|---|---|---|---|---|---|---|---|
| | | 5 | 2 | 8 | 12 | 20 | 11 |
| T | C. | 143 | 115 | 143 | 20 | 20 | 130 |
| Mass flow rate | kg/hr | 6500 | 13 850 | 1350 | 300 | 500 | 447 |
| Mass fraction | | | | | | | |
| AA | | 0.000 | 0.066 | 0.000 | 0.118 | 0.000 | 0.000 |
| E2HOH | | 0.000 | 0.233 | 0.000 | 0.110 | 0.000 | 0.000 |
| 2EHA | | 0.953 | 0.651 | 0.786 | 0.729 | 0.000 | 0.487 |
| HPA | | 0.000 | 0.000 | 0.000 | 0.000 | 0.000 | 0.000 |
| WATER | | 0.000 | 0.005 | 0.000 | 0.010 | 1.000 | 0.000 |
| DIAA | ppm | 481.0 | 224.0 | 220.0 | 0.0 | 0.0 | 2522.0 |
| 2EHEXENE | ppm | 0.0 | 1872.0 | 0.0 | 2991.0 | 0.0 | 0.0 |
| HPA | ppm | 47.0 | 71.0 | 15.0 | 2991.0 | 0.0 | 15.0 |

| | Stream | | | | |
|---|---|---|---|---|---|
| | 7 | 4 | 21 | 10 | 19 |
| T | 24 | 30 | 20 | 35 | 20 |
| Mass flow rate | 5150 | 7919 | 500 | 903 | 300 |
| Mass fraction | | | | | |
| AA | 0.000 | 0.119 | 0.032 | 0.000 | 0.065 |
| E2HOH | 0.000 | 0.420 | 0.000 | 0.000 | 0.110 |
| 2EHA | 0.997 | 0.405 | 0.000 | 0.934 | 0.728 |
| HPA | 0.000 | 0.000 | 0.000 | 0.000 | 0.000 |
| WATER | 0.000 | 0.011 | 0.966 | 0.000 | 0.068 |
| DIAA | 31.0 | 4.0 | 0.0 | 2035.0 | 0.0 |
| 2EHEXENE | 0.0 | 4420 | 52.0 | 0.0 | 29 786.0 |
| HPA | 55.0 | 93.0 | 1725.0 | 16.0 | 118.0 |

### Example Laboratory Tests 1: Continuous Cracking

The thermosiphon reactor is fed continuously using a diaphragm pump.

The thermosiphon is mounted with a condenser so as to recover the vapors of the light products formed.

A vacuum pump is connected to the outlet of the serpentine condenser and also to the pot collecting the heavy products or residues.

The heavy product stream is first preheated to 120° C. before reaching the reactor, so as to keep the stream in its liquid form. The apparent residence time (thermosiphon volume/entering volumetric flow rate) is 4.5 h, the pressure is 50 000 pascals and the bottom temperature is 245° C.

The initial feed contains the compounds listed in Table 6. No catalyst is added to this feed.

TABLE 6

| | Feed |
|---|---|
| 2-ethylhexanol | 0.9 |
| 2EHA | 22.5 |
| 2EHHP | 7.8 |
| 2EHAP | 20.8 |
| OPO | 16.3 |
| % H2O | 1.2 |
| HPA (value) | 0.2 |
| AA | 0.0 |
| AA2 | 0.6 |
| HOME | 0.1 |
| PTZ | 1.4 |
| PTZ/AA | 0.1 |
| PTZ/2EHA | 2.6 |

The depletion ratio (head flow rate/feed flow rate) is 70% on average. The main adduct cracking contents are 96% for 2EHHP, 85% for 2EHAP and 56% for OPO. The composition of the head stream is indicated in Table 7.

TABLE 7

| Head mass % | Head |
|---|---|
| Sum of C8 olefins | 0.1-0.2 |
| 2-ethylhexanol | 9.4-10.8 |
| 2EHA | 63.1-72 |
| 2EHHP | 0.7-1.8 |
| 2EHAP | 1.5-3.5 |
| OPO | 0.3-5 |
| HPA | 0.1-0.2 |
| AA | 6.9-10.9 |
| HOME | 0.2-0.4 |
| WATER | 1.5-2.4 |

This stream contains between 79% and 94% of upgradable compounds. It also contains very few olefins in the absence of catalyst. It contains 0.1% to 0.2% of HPA, which must then be removed via the process according to the invention.

The bottom stream composition is shown in Table 8.

TABLE 8

| Residue mass % | Residue |
|---|---|
| 2-ethylhexanol | 0.5-0.6 |
| 2EHA | 4.4-5 |
| 2EHHP | 0.5-0.7 |
| 2EHAP | 6.6-7.3 |
| OPO | 23.6-29.6 |
| AA | 0.2 |
| PTZ | 2.9-7.4 |
| PTZ/2EHA | 0.8-1.9 |
| Viscosity Poises | 0.3-0.8 |

The bottom residue contains less than 5% of 2EHA and less than 40% of adducts. The other very heavy compounds (about 45%) cannot be analyzed by gas chromatography. Its viscosity is very low and it contains no solids that would result in clogging of the facility.

### Example Laboratory Tests 2: HPA Extraction from a Cracker Head Stream

The cracker head stream is washed as follows.

It is mixed with the same mass of water at room temperature.

Table 9 shows the composition of two cracker head streams. They contain 0.3% and 0.08% of HPA and nearly 90% of upgradable compounds.

TABLE 9

| mass % | Head 1 | Head 2 |
|---|---|---|
| HPA | 0.324 | 0.082 |
| Sum of C8 olefins | 0.97 | 0.75 |
| 2-ethylhexanol | 12.9 | 13.7 |
| 2EHA | 64.9 | 63.2 |
| 2EHHP | 0.79 | 0.66 |
| 2EHAP | 1.96 | 1.75 |
| OPO | 2.22 | 2.11 |
| AA | 11.9 | 13.6 |
| WATER | 2.1 | 2.1 |

| | Washed | Washed |
|---|---|---|
| Mass % composition | head 1 | head 2 |
| Final HPA | 0.016 | 0.001 |
| Sum of C8 olefins | 1.0 | 0.8 |
| 2-ethylhexanol | 13.4 | 14.9 |
| 2EHA | 67.8 | 68.6 |
| 2EHHP | 0.8 | 0.7 |
| 2EHAP | 2.0 | 1.9 |
| OPO | 2.3 | 2.3 |
| AA | 8.3 | 6.8 |
| WATER | 2.4 | 2.1 |

HPA is extracted to more than 94% and 99% from the organic phase in only one separation stage. The degree of AA extraction is 25% and 55%, respectively.

1. A process for recovering/purifying a C₄-C₁₀ acrylic ester from a crude reaction mixture obtained via direct esterification of acrylic acid with a corresponding alcohol, in which a stream rich in acidic impurities originating from the esterification reaction, are withdrawn via a side outlet during distillation of the crude reaction mixture, said process comprising a step of thermal cracking of residues at a bottom of a purification column, leading to production of cracking products which are recycled into the process.

2. The process as claimed in claim 1, in which said recycled cracking products are olefins, acidic impurities, the ester and reagents.

3. The process as claimed in claim 1 in which cracking is performed in the absence of catalyst.

4. The process as claimed in claim 1 wherein a stream rich in acidic impurities, drawn off laterally, is subjected to treatment with water, and the treated stream is recycled into a distillation column.

5. A process for the recovery/purification of a C₄-C₁₀ acrylic ester from a crude reaction mixture obtained by direct esterification of acrylic acid with a corresponding alcohol, comprising at least the following steps:
i) subjecting the reaction mixture to topping in a distillation column equipped with a side draw-off to obtain:
at the top, a stream essentially comprising unreacted reagents;
at the bottom, a stream comprising desired ester and heavy byproducts;
by side draw-off, a stream rich in acidic impurities;
ii) subjecting the bottom stream from the topping column to a rectification column to separate:
at top, purified desired ester;
at bottom, a stream containing heavy byproducts, which is concentrated on a film evaporator or distilled in a tailing column so as to recycle light compounds present to the topping column, and to eliminate a final residue of heavy byproducts;
iii) subjecting the bottom stream of the rectification column to a thermal or thermal and catalytic treatment performed in a cracker placed at an outlet of the evaporator, to separate:
at top, a stream of upgradable products recycled separately or mixed with evaporator head stream;
at bottom, a residue sent to a treatment plant.

6. The process as claimed in claim 5 further comprising a step iv); treating the stream drawn off laterally, in which the stream rich in acidic impurities is subjected to a washing step with an aqueous stream, which produces, after decantation:
an aqueous phase comprising all of the acidic impurities, which can be sent to a biological treatment plant or partly used as aqueous washing stream, and
an organic phase comprising desired ester, heavy byproducts and traces of water and reagents, which is at least partly recycled into the topping column.

7. The process as claimed in claim 6, further comprising a step v) treating cracker head stream:
subjecting the cracker head stream to a step of washing with an aqueous stream, which produces, after decantation:
an aqueous phase comprising all of the acidic impurities, which can be sent to a biological treatment plant or partly used as aqueous washing stream, and
an organic phase, comprising the desired ester, the alcohol and acid reagents, heavy byproducts and traces of water, which is at least partly recycled into the topping column feed or into the side draw-off washing step.

8. The process as claimed in claim 5 in which the cracker is a tubular reactor, a jacketed stirred reactor or a reactor with an external heating loop with forced circulation or natural circulation.

9. The process as claimed in claim 5 in which a decomposition reaction is performed in a temperature range of from 180 to 280° C.

10. The process as claimed in claim 5 in which cracker head products are washed in a decanter with water and then mixed with evaporator head product to be recycled to an inlet of the topping column.

11. The process as claimed in claim 5 in which cracker head products are washed in a decanter with water and then introduced into a decanter present at the side draw-off.

12. The process as claimed in claim 5 in which cracker head products are introduced directly into a decanter present at the side draw-off.

13. The process as claimed in claim 1 wherein the C₄-C₁₀ acrylic ester is 2-ethylhexyl acrylate.

14. A process for producing a C₄-C₁₀ acrylic ester free of acidic impurities by direct esterification of acrylic acid with the corresponding alcohol, comprising the recovery/purification process as defined in claim 1.
